(19) United States
(12) Patent Application Publication
(10) Pub. No.: US 2022/0211557 A1
(43) Pub. Date: Jul. 7, 2022

(54) TRAINING PANTS WITH GRAPHICS

(71) Applicant: FIRST QUALITY BABY PRODUCTS, LLC, Great Neck, NY (US)

(72) Inventors: Joseph Gatta, Newtown, PA (US); Michael S. McKee, Downing ton, PA (US); Heidi Marie Warner, Mill Hall, PA (US)

(21) Appl. No.: 17/569,569

(22) Filed: Jan. 6, 2022

Related U.S. Application Data
(60) Provisional application No. 63/134,660, filed on Jan. 7, 2021.

(51) Int. Cl. A61F 13/84; A61F 13/49; A61F 13/496

(52) U.S. Cl. CPC A61F 13/84; A61F 2013/8497; A61F 13/496; A61F 13/49015

(57) ABSTRACT

Training pants including a chassis having an absorbent core, a backsheet, and a cover, and elastic side panels attached to the chassis. The training pants include a first graphic on a external perimeter of the training pants, a second graphic in a middle portion of a front and/or back surface of the training pants, and a third graphic on the front and/or back surface of the training pants.

### RELATED APPLICATIONS

This application claims priority to and the benefit of U.S. Provisional Application No. 63/134,660, entitled TRAINING PANTS WITH GRAPHICS filed Jan. 7, 2021, the contents of which are incorporated herein by reference in their entirety.

### FIELD OF THE INVENTION

The present invention generally relates to training pants, and in particular to training pants with graphics.

### BACKGROUND

Training pants have been made and sold for many years. In general, babies wear diapers to prevent accidental contact with urine or feces. Diapers have hook and loop tabs on front and back panels. When a diaper is placed on the baby, the hooks and loops are placed in contact with one another and attach to hold the diaper on the baby. To change the diaper, the care-giver pulls the hooks and loops apart.

Parents typically begin to potty train toddlers to switch them to underwear. Technology and materials have evolved such that training pants were developed and have become a growing market. Training pants are generally made by forming an insert/core, moving the insert/core in a machine direction, and then applying it to side panels, which typically include elastic materials for stretchability.

Conventional training pants are greatly improved in that the hooks and loops may be attached on the side of the training pants. The child may pull the training pants down to use the potty, then pull them back up. It is also easier for a parent to check the training pant to see if a change is necessary.

Toddlers often do not like wearing training pants because they are more like diapers than underwear. For this and other reasons, there is a continuing need to provide improved training pants.

### SUMMARY OF THE INVENTION

Training pants according to an exemplary embodiment of the present invention comprise: a chassis comprising an absorbent core, a backsheet, and a cover; and two elastic side panels attached to the chassis, wherein the training pants include: a first graphic on an external perimeter of the training pants; a second graphic in a middle portion of a front and/or back surface of the training pants; and a third graphic on the front and/or back surface of the training pants.

In an exemplary embodiment, the first, second, and third graphics are different from one another in terms of type of graphic.

In an exemplary embodiment, the first graphic comprises a pattern that provides an appearance of a structural element, such as lacing or stitching.

In an exemplary embodiment, the second graphic is an image of an object.

In an exemplary embodiment, the third graphic comprises wording that forms a phrase.

### BRIEF DESCRIPTION OF THE DRAWINGS

FIG. 1 is a top view of a training pant according to an exemplary embodiment of the present invention.

### DETAILED DESCRIPTION OF PREFERRED EMBODIMENTS

Training pants according to exemplary embodiments of the present invention include a chassis made up of an absorbent core or insert, a topsheet, a backsheet and two elastic side panels.

Absorbent cores or inserts may be formed or cut out from rolls of absorbent materials. As used herein, the term “absorbent core or insert” refers to a material or combination of materials suitable for absorbing, distributing, and storing aqueous fluids such as urine, blood, menses, and water found in body exudates. The size and shape of the absorbent core can be altered to meet absorbent capacity requirements; and to provide comfort to the wearer/user. The absorbent core suitable for use in the present invention can be any liquid absorbent material known in the art for use in absorbent articles, provided that the liquid absorbent material can be configured or constructed to meet absorbent capacity requirements. Nonlimiting examples of liquid absorbent materials suitable for use as the absorbent core include comminuted wood pulp, which is generally referred to as airfelt; creped cellulose wadding; absorbent gelling materials including superabsorbent polymers, such as hydrogel-forming polymeric e g agents; chemically, stiffened, modified, or cross-linked cellulose fibers; meltblown polymers including coform; synthetic fibers including crimped polyester fibers; tissue including tissue wraps and tissue laminates; capillary channel fibers; absorbent foams; absorbent sponges; synthetic staple fibers; peat moss; or any equivalent material; or combinations thereof, as is well known in the art of making absorbent products such as sanitary napkins, pantiliners, incontinence pads; and the like.

The core or insert is placed on a top surface of a backsheet. Backsheets are materials that generally are liquid impermeable, but may be moisture vapor permeable (breathable). Backsheets are used in absorbent products on a surface of the product that is distal to the user's body. The backsheet can be any known or other effective backsheet material, provided that the backsheet prevents external leakage of exudates absorbed and contained in the protective underwear. Flexible materials suitable for use as the backsheet include, but are not limited to, woven and nonwoven materials, laminated tissue, polymeric films such as thermoplastic films of polyethylene and/or polypropylene, microporous films, composite materials such as a film-coated nonwoven material, or combinations thereof, as is well known in the art of making absorbent products, such as sanitary napkins, pantiliners, incontinence pads; and the like.

The absorbent core or insert is typically attached to the backsheet with an adhesive. Suitable adhesives are known in the art and include hot melt adhesives, emulsion polymer adhesives and the like.

A topsheet or cover is placed on top of the core or insert and attached to the core or insert and backsheet with adhesive, ultrasonic bonding or combinations thereof, forming a chassis. Suitable topsheets are compliant, soft feeling, and non-irritating to the body of the wearer. Suitable topsheet materials include a liquid pervious material that is oriented towards and contacts the body of the wearer, thereby permitting body discharges to rapidly penetrate through the topsheet without allowing fluid to flow back through the topsheet to the skin of the wearer. A suitable topsheet can be made of various materials, such as woven and nonwoven materials; apertured film a materials including apertured formed thermoplastic films, apertured plastic films, and fiber-entangled apertured films; hydro-formed thermoplastic films; porous foams; reticulated foams; reticulated thermoplastic films; thermoplastic scrims; or combinations thereof, as is well known in the art of making absorbent products such as sanitary napkins, pantiliners, incontinence pads, protective and the like.

The chassis may include additional materials such as acquisition distribution layers, transfer layers, secondary absorbent layers and the like.

Elastic side panels are attached to the chassis to form training pants. Any elastic side panel known in the art of absorbent articles may be useful. Suitable elastic side panels include laminates of elastic films with nonwovens, laminates of elastic strands with nonwovens and the like. The elastic panels may be attached to the chassis by adhesive, ultrasonic bonding or a combination thereof. The length, width and shape of the side panels may be designed to make training pants of different sizes. The training pants have a more underwear like appearance.

The elastic side panels may include two front side panels that can be coupled to two back side panels forming a seam for securing the training pants to the waist of the wearer. Typical seams are centered on a side portion of the training pant, and can be refastenable, non-refastenable (i.e., “sealed”), or a combination thereof. Refastenable seams can be formed by using fastening components, such as hook and loop fasteners. Hook and loop fasteners may be made up of separate hook and loop elements, or may be integral with the side panels. When overlapping refastenable seams are included, training pants may include hook elements on inside or outside surfaces of the side panels. In exemplary embodiments, the chassis includes front and back waist portions, and the hook elements may directly engage with the front and/or back waist portions rather than corresponding side panels having loop elements. In this regard, the training pant may be “loopless” in that loop elements are not included and instead the hook elements directly engage with the front waist portion, the back waist portion, and/or corresponding side panels that do not include hook elements.

An absorbent article according to an exemplary embodiment of the present invention includes side panels having integrated hook elements. In this regard, the hook elements are intimately joined and form a unitary structure with the material that forms the side panels. Such intimate bonding of hook elements with a layer of material may be accomplished by feeding extruded plastic and a sheet of material through a nip formed by a first roller having pins and a second roller having corresponding cavities. As the molten plastic is forced through the nip, it flows into the cavities of the second roller and also into pores in the sheet of material. The plastic in the cavities cools and hardens so as to form a hook sheet. At the same time, the sheet of material is intimately joined to and becomes an integral part of the hook sheet so as to form a laminated structure. This process is disclosed in U.S. Pat. No. 5,518,795, the contents of which are incorporated herein by reference in their entirety. The hook elements may be arranged on the side panels in longitudinally extending strips that are laterally spaced from one another. Alternatively, the hook elements may be arranged in a pattern of geometric shapes or lines. Desirably, the hook elements are arranged on an inelastic material in order to improve ease of processing and the shear strength of the seam.

In exemplary embodiments, lateral end portions of the front side panels are reduced in length relative to remaining portions of the front side panels so as to form tabs. The tabs laterally extend beyond the hook elements so that a user may grasp the tabs when separating the side seams.

The training pants may be made by forming or cutting an absorbent core or insert. The core or insert is placed on a backsheet and fed in a machine direction to a cover placement module, where a cover or topsheet is attached to the core and backsheet by adhesive, ultrasonic bonding or a combination thereof, forming a chassis. The chassis is fed to an elastic side panel application module where elastic side panels are attached along edges of the chassis by adhesive, ultrasonic bonding or a combination thereof.

The two side panels are then attached to the insert/core, with each side panel attached to the leading and trailing edges portions as well as respective side edge portions of the insert/core. In embodiments, a portion of each side panel is left unattached to the respective side edge portion of the insert/core to form leg openings. The side panels may be attached to the insert/core with glue, adhesive, ultrasonic bonding or combinations thereof. The side panels may be attached to the insert/core at various angles to create a more garment like fit.

Side panel web paths may go through a re-pitch unit to allow for additional material longer than the insert/core length to be applied per product. Waist diameters can be modified by adjusting the re-pitch length which obviates the need to add additional costly materials to make the insert/core longer. Some re-pitch machines include two pucks operating at different speeds which pick up, move and place material on an insert/core. Suitable re-pitch devices and methods are taught in U.S. Pat. Nos. 9,498,941 and 9,827,147, the contents of which are hereby incorporated by reference in their entirety. In prior processes, the product geometry for the waist is dictated by the insert/core length, creating a costly product. The method of the present invention allows over feed side panel webs so a longer side panel may be attached to the same chassis, which enables making a different size waist.

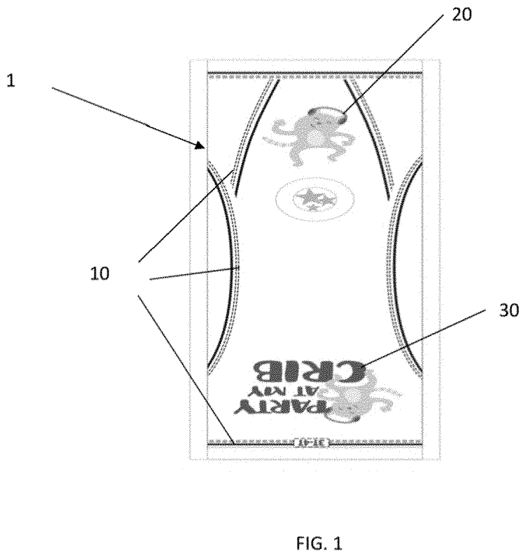

FIG. 1 shows training pants, generally designated by reference number 1, according to an exemplary embodiment of the present invention. As shown in FIG. 1, the training pants 1 include a first graphic 10 on the external facing perimeter of the training pants. The first graphic 10 may be similar to lace or stitching on underwear. The design may be applied by printing ink using techniques such as gravure, flexographic, roll printing and the like. The color of the ink is not critical but may be selected from blue, green, red, black, white, yellow, orange and the like. In exemplary embodiments, the first graphic may extend continuously at least along the leg openings and back and front waist portions of the training pants 10 so that those portions of the training pants appear to have lace, stitching, or some other structural elements or combinations thereof.

The training pants 10 also include a second graphic 20 in a middle portion of a front and/or back external surface of the training pants. The second graphic 20 may be an image of an object, where the object may be, for example, a dog, cat, bear, cow, deer, chicken, bunny, bird, fox, giraffe, turtle, pig, penguin, sippy cup, baby bottle, home, and the like or combinations thereof. The second graphic 20 may be applied by printing ink using techniques such as gravure, flexographic, roll printing and the like. The color of the ink is not critical but may be selected from blue, green, red, black, white, yellow, orange and the like or combinations thereof.

The training pants include a third graphic 30 on the front and/or back external surface of the training pants. The third graphic 20 may be wording, such as, for example, a combination of letters and words that make up a phrase, where the phrase may be, for example, “popping bottles”, “I still live with my parents”, “what's shakin, bacon”, “party at my crib”, “there's a nap for that”, “I'm kind of a big deal”, “Oh my deer”, “Let's cuddle”, and the like. The third graphic 30 may be applied by printing ink using techniques such as gravure, flexographic, roll printing and the like. The color of the ink is not critical but may be selected from blue, green, red, black, white, yellow, orange and the like or combinations thereof.

In embodiments, the wording of the third graphic 30 may be a caption corresponding to the object of the second graphic 20. For example, combinations of the second and third graphics 20, 30 may include, but are not limited to a pig with “what's shakin', bacon”; a bear with “unbearably cute”; a house with “I still live with my parents”; baby bottles or sippy cups with “poppin' bottles”; a panda bear with “there's a nap for that”; balloons with “I'm kind of a big deal”; a deer with “oh my deer”; a bear with “let's cuddle” or other combinations thereof.

Training pants of the present invention provide aesthetically appealing products that promote compliance with potty training. The toddler will be enticed to wear the product and more likely to wear the training pants during training. The toddler will also feel more grown up (like a big kid) because the training pants look more like adult underwear as opposed to a diaper. The toddler will also receive positive reinforcement when adults compliment them on the training pants and progress with potty training.

Now that embodiments of the present invention have been shown and described in detail, various modifications and improvements thereon can become readily apparent to those skilled in the art. Accordingly, the exemplary embodiments of the present invention, as set forth above, are intended to be illustrative, not limiting. The spirit and scope of the present invention is to be construed broadly.

We claim:

1. Training pants comprising:
i. a chassis comprising an absorbent core, a backsheet, and a cover; and
ii. two elastic side panels attached to the chassis, wherein the training pants comprise:
a first graphic on an external perimeter of the training pants;
a second graphic in a middle portion of at least one of a front or back external surface of the training pants; and
a third graphic on at least one of the front or back external surface of the training pants.

2. The training pants according to claim 1, wherein the first graphic comprises a pattern that provides an appearance of a structural element.

3. The training pants according to claim 2, wherein the structural element is lacing or stitching.

4. The training pants according to claim 1, wherein the second graphic comprises an image of an object.

5. The training pants according to claim 4, wherein the object is selected from the group consisting of a dog, cat, bear, cow, deer, chicken, bunny, bird, fox, giraffe, turtle, pig, penguin, sippy cup, baby bottle, home and combination thereof.

6. The training pants according to claim 1, wherein the third graphic comprises wording.

7. The training pants according to claim 6, wherein the wording comprises a phrase.

8. The training pants according to claim 1, wherein the second graphic comprises an image of an object and the third graphic comprises wording.

9. The training pants according to claim 8, wherein the wording is a caption corresponding to the object.
